(19) United States
(12) Patent Application Publication
(10) Pub. No.: US 2013/0144300 A1
(43) Pub. Date: Jun. 6, 2013

(54) APPLICATOR KIT

(71) Applicants: Sebastian Vogt, Erfurt (DE); Daniela Villone, Bad Homburg (DE); Frank Finis, Augsburg (DE); Rüdiger Creuzburg, Weimar (DE)

(72) Inventors: Sebastian Vogt, Erfurt (DE); Daniela Villone, Bad Homburg (DE); Frank Finis, Augsburg (DE); Rüdiger Creuzburg, Weimar (DE)

(73) Assignee: HERAEUS MEDICAL GMBH, Wehrheim (DE)

(21) Appl. No.: 13/553,305

(22) Filed: Jul. 19, 2012

(30) Foreign Application Priority Data
Jul. 20, 2011 (DE) 10 2011 108 010.8

(51) Int. Cl. A61F 2/28

(52) U.S. Cl. 606/94

(57) ABSTRACT

An applicator and applicator kit for particulate bone replacement materials, autologous or heterologous cancellous bone, demineralised bone matrix. The applicator (1) contains a hollow tube (12) including on one end a bladed shutter. The applicator comprises first fastener (15) for engaging a second component. The second component contains a rod (2) with a handle (21) on one end and a plunger (22) on the opposite end. The rod is arranged in a guide sleeve which is moved along the rod (2) and comprises second fastening means (23) that can be connected to the fastener (15) of the applicator (1) in reversible manner. The applicator kit contains a mixing cup connected to the tube (12) instead of the rod such that the material to be applied is filled into the tube (12) easily prior to application.

### BACKGROUND OF THE INVENTION

(1) Field of the Invention

The invention relates to an applicator for application of particulate bone replacement materials, autologous or heterologous cancellous bone, demineralised bone matrix or mixtures thereof as well as an applicator kit that contains said applicator.

(2) Description of Related Art

It is still customary to fill bone defects with autologous cancellous bone, However, only relatively small volumes of autologous cancellous bone can be harvested from the patient. This limits the use of autologous cancellous bone despite its good osseointegration behavior. One possible solution to this issue is to augment the autologous cancellous bone with synthetic or semi-synthetic bone replacement materials. The most commonly used bone replacement materials currently are calcium phosphates, such as β-tricalcium phosphate, hydroxyl apatite, and mixtures thereof. These are generally present as porous or non-porous granules. Besides, particulate bone replacement materials based on calcium sulfate are known also.

It is customary to mix autologous cancellous bone and particulate bone replacement materials in the operating theatre by simply mixing the cancellous bone and the bone replacement material in a sterile vessel,

Moreover, a number of devices for mixing bone replacement materials with cancellous bone or blood or platelet-rich plasma (PRP) have been described.

WO 2002/068010 A1 proposed a device, in which aspirated bone marrow can be mixed easily with a porous bone replacement material. The aspirated bone marrow is pressed or aspirated through the bone replacement material using two syringes.

A device with a similar effect is known from U.S. Pat. No. 6,049,026 A1. Said device consists of a container that takes up the bone replacement material and a second container that is arranged above said container. The purpose of the second container is to take up the aspirated bone marrow. Moreover, a third container is arranged below the first container. A second valve is arranged between the first and the second container. Another valve is situated between the first and the third container. After the first valve is opened, the aspirated bone marrow flows into the first container and soaks the bone replacement material. Then, the second valve is opened and the excess bone marrow flows into the third container.

A very simple device has been disclosed in U.S. Pat. No. 7,776,594 B2. The mixing container consists of a tube with two screw caps at the ends. The bone replacement material is simply introduced into the tube and then the aspirated bone marrow is added by injection.

U.S. Pat. No. 6,793,660 B2 described a device for introducing bone replacement materials and consists of a syringe with an internal thread and a rotatable plunger with an external thread. The bone replacement material is simply squeezed out in the direction of the syringe head through rotating the plunger. One problem is the very large dispensing force of said system since this may mechanically damage granular bone replacement material. Moreover, where autologous cancellous bone or mixtures of autologous cancellous bone and bone replacement materials are used, it is possible that cancellous bone cells might be damaged when they are exposed to the strong force.

A recurring problem in the operating theatre is that particulate bone material or mixtures thereof that include cancellous bone or blood or platelet-rich plasma are difficult to introduce using just spoons, spatulas or modified syringes whose syringe head has been cut off. In particular, the presence of cavities in long tubular bones is a problem. It would therefore be expedient to have a long, syringe-like applicator. Moreover, filling mixtures that include blood or blood components into modified syringes through the use of spoons and syringes without contaminating the OR staff and the surgical field is associated with a hygiene problem. Thus far, no simple, safe and hygienic transfer of bone replacement materials or mixtures thereof from mixing containers to suitable syringe-like applicators is known.

### BRIEF SUMMARY OF THE INVENTION

The invention is based on the object to develop a simple applicator kit for application of particulate bone replacement materials, autologous or heterologous cancellous bone, demineralised bone matrix or mixtures thereof, which kit overcome the disadvantages of the prior art.

The object was met by providing an applicator for bone replacement material comprising a hollow tube which has a bladed shutter at a front end and is equipped with first fastening means on a rear end, and a rod that comprises a handle on one end and a plunger on the opposite end, and is arranged inside a guide sleeve that can be moved along the rod, whereby the guide sleeve has a diameter that is smaller than or equal to the internal diameter of the tube, and second fastening means that can be connected. The object is further met by providing an applicator kit by providing an applicator and a mixing cup that possesses at its underside at least one opening that is surrounded by a dispensing tube on its external side, whereby the dispensing tube has a diameter which is smaller than or equal to the internal diameter of the widened section, and comprises a third fastening means that can be connected to the fastening means of the applicator in reversible manner.

The invention relates to an applicator comprising a hollow tube that has a bladed shutter on one end and first fastening means on the other end, a rod that comprises a handle on one end and a plunger on the opposite end, and is arranged inside a guide sleeve that can be moved along the rod, whereby the guide sleeve has a diameter that is smaller than or equal to the internal diameter of the hollow tube, and second fastening means that can be connected to the fastening means of the applicator in reversible manner.

The invention also relates to an applicator kit containing an applicator according to any one of the preceding claims and a mixing cup that possesses at its underside at least one opening that is surrounded by a second sleeve on its external side, whereby the second sleeve has a diameter than is smaller than or equal to the internal diameter at the rear end of the tube, and comprises third fastening means that can be connected to the fastening means of the applicator in reversible manner.

In the scope of the invention, “rear end” of the applicator shall be understood to refer to the end at which the rod is being introduced, whereas “front end” shall be understood to refer to the end provided with the bladed shutter, at which the granulate bone replacement material is being squeezed out.

As a matter of principle, the applicator tube can be conceived to have any of various cross-sections, but a tube having a circular cross-section is preferred. Likewise, the widened section may also have various cross-sections. The cross-section of the widened section does not necessarily have to be of the same shape as the cross-section of the tube, but the area of the cross-section is larger than that of the tube. Moreover, it is advantageous for the cross-section to be circular.

Rod and tube are arranged in the applicator much like in a medical syringe. However, the two design elements are fixed to each other through fastening means. A wide range of fastening means can be applied in this context. A bayonet catch has proven to be particularly advantageous, since it can be closed rapidly and can be released just as rapidly. However, other fastening means are conceivable as well, for example a screw closure.

The fastening means are arranged at the rear end of the tube and on a guide sleeve of the rod. The rod can be moved back and forth inside the guide sleeve, Preferably, the rear end of the tube is widened and forms a cylinder section having a larger diameter than the tube. In this case, the guide sleeve preferably has an external diameter that corresponds to the internal diameter of the widening or is slightly smaller, The fastening means are then attached on the external circumference of the guide sleeve and on the internal circumference of the cylinder section.

The bladed shutter is ideal for dispensing a mass such as autologous cancellous bone. Only when pressure is being applied to the mass, the mass presses the blades outwards and dispenses the bone replacement material. It is preferable for the blades to form a tip. The distance between the blades should be adapted to the granulate and should be sufficiently small in the closed state for the material to not exit. Bending open the blades produces an opening that is sufficiently large for the material to exit from it. It shall therefore be understood that “closed state” does not necessarily mean that the blades form a liquid- or gas-tight closure, but rather it means that the bone replacement material cannot exit.

Preferably, the hollow tube of the applicator has an external diameter of 4 to 15 mm, particularly preferably of 8 to 13 mm, and even more particularly preferably of 11 to 13 mm. The length from the front end including bladed shutter to the end of the cylinder-shaped widening preferably is from 20 to 40 cm, particularly preferably is from 25 to 35 cm, and even more particularly preferably is from 28 to 32 cm.

Accordingly, the design of the applicator in terms of its length and diameter is such that it allows the applicator to be introduced into tubular bones, such as tibia and femur, without any trouble and then allows particulate material to be introduced safely, without any trouble, even into the cavities of tubular bones that are farthest from the access, thus enabling these to be filled to the largest extent possible.

Preferably, the applicator is made of a material with some elasticity. The materials should be biocompatible, such as, for example, polypropylene. Selecting an elastic material allows the applicator to follow the anatomy-dependent curvatures of tubular bones while it is being inserted.

Preferably, a mixing spoon or mixing spatula is arranged in the mixing vessel, whereby it is particularly preferred to have a mixing spatula possessing on the end of the spatula a two-dimensional stopper that is situated perpendicular to the axis of the mixing spatula and whose cross-sectional area is smaller than or equal to the cross-sectional area of the opening of the mixing cup.

The application of particulate bone replacement materials, autologous or heterologous cancellous bone, demineralised bone matrix or mixtures thereof through the use of the applicator kit proceeds by first filling the particulate bone replacement materials, autologous or heterologous cancellous bone, demineralised bone matrix or mixtures thereof into the mixing cup. The slider doses the opening of the mixing cup during the process. The mixing cup is then introduced into the cylinder-shaped widening using the connector, and the fastening means are connected to each other in reversible manner. Subsequently, the slider is pulled such that the opening of the mixing cup is exposed and the material drops into the hollow tube of the applicator. If applicable, the material can just as well be stuffed into the hollow space of the applicator using the stopper of the mixing spatula. Subsequently, the connection between the applicator and the mixing cup is released through release of the fastening means. Then, the rod with guide sleeve is inserted such that the guide sleeve is situated in the widening of the applicator. The fastening means on tube and rod are made to engage each other. Sliding the rod causes the material to be pushed in the direction of the bladed shutter, the bladed shutter bends open, and the material is dispensed.

The applicator kit according to the invention affords numerous advantages as compared to the prior art.

Prior to the actual application, the applicator kit allows particulate bone replacement materials to be mixed with autologous or heterologous cancellous bone or demineralised bone matrix and to be transferred cleanly and safely to the applicator.

The applicator can be actuated easily and safely by hand. Simply pressing against the rod allows the bone replacement material to be squeezed from the applicator without the particulate bone replacement material being damaged by shearing at sharp edges.

By this means, some problems that are inherent to the use of conventional applicators are prevented. The bone material is safely taken up by the application tube and retained by the bladed shutter such that the bone replacement material cannot inadvertently exit from the applicator and thus contaminate the surgical field prior to the actual application. Having to remove granulate material from the soft tissue surrounding the bone would disturb the surgery and unnecessarily prolong the duration of the surgery. Moreover, the rod being fixed in the tube prevents the rod from dropping into the surgical field or onto the floor of the operating theatre, which would also disturb the progress of the surgery.

### BRIEF DESCRIPTION OF THE SEVERAL VIEWS OF THE DRAWING(S)

The invention shall be described in the following in exemplary fashion based on the appended drawings. In the figures:

FIG. 1 shows a preferred embodiment of the applicator in the form of a detail view and a partial sectional view;

FIG. 2 shows another detail view of the applicator; and

FIG. 3 shows a perspective view of a mixing cup of an applicator kit.

### DETAILED DESCRIPTION OF THE INVENTION

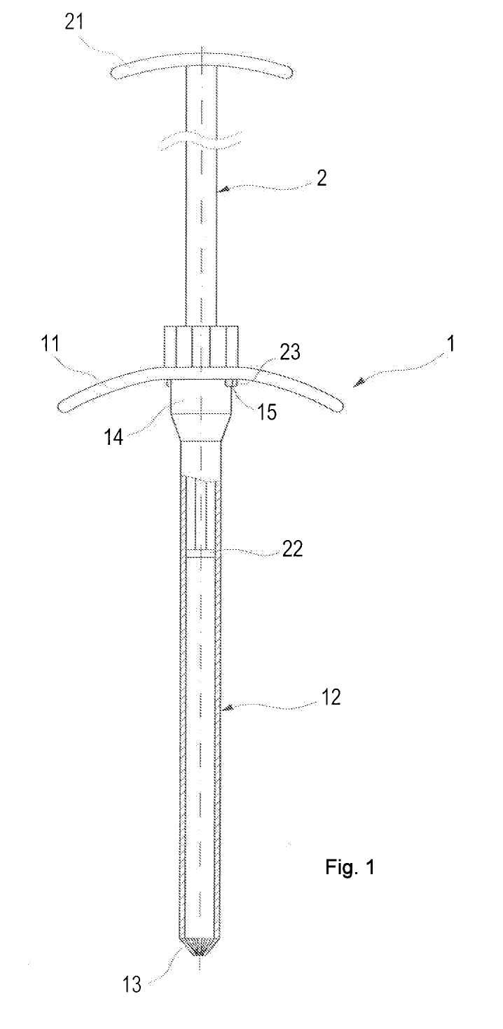

FIG. 1 shows a partial sectional view of the applicator 1. It comprises a hollow tube 12, in which the autologous cancellous bone is situated. The tube 12 has, at its rear end, a widened cylindrical section 14, into which a guide sleeve is inserted. The purpose of the guide sleeve is to guide a rod 2 to which a plunger 22 is fastened. The guise sleeve comprises a pin 23 that acts in concert with an L-shaped recess 15 in the widened cylindrical section 14.

By applying pressure to the rod 2, the plunger 22, which is arranged on the rod, is shifted forward and the autologous cancellous bone is pressed against a bladed shutter 13. The bladed shutter 13 is made of an elastic material such that the individual blades are bent outwards when the cancellous bone is pressed against the blades.

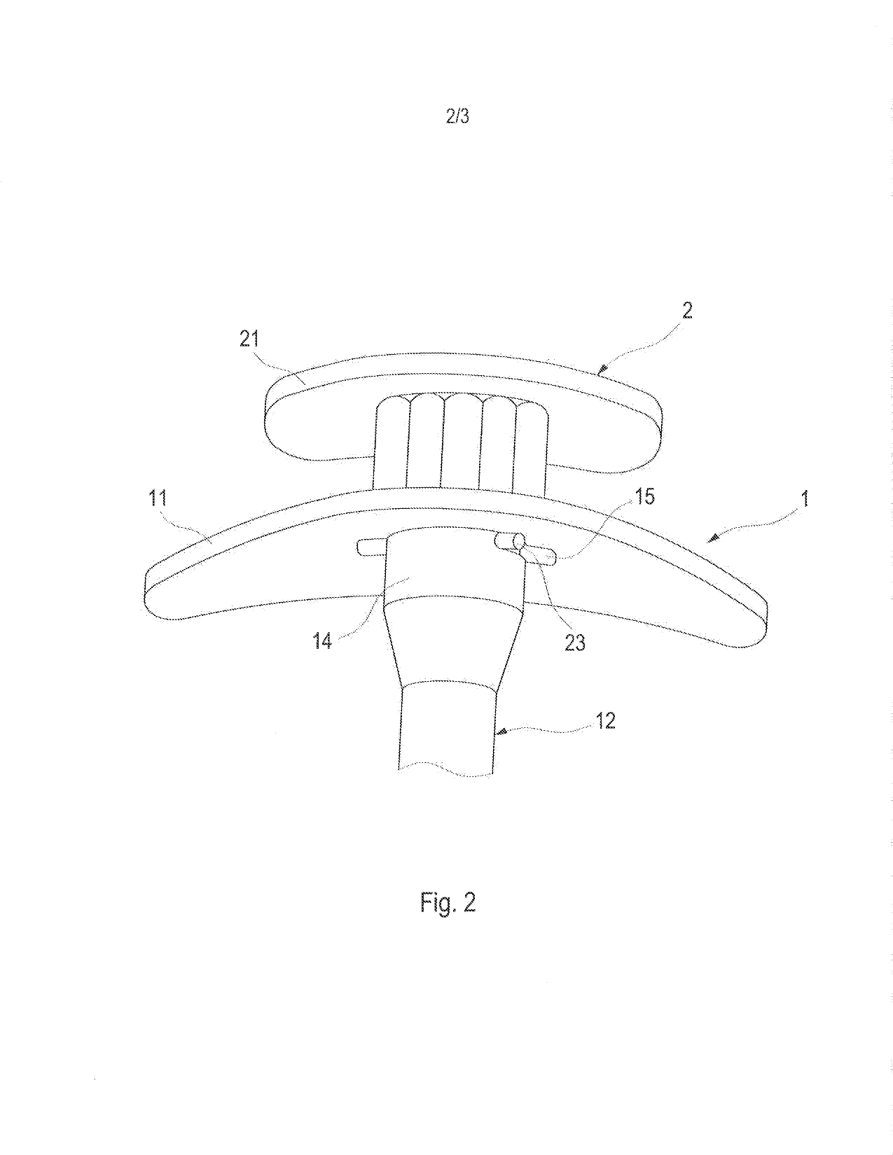

FIG. 2 shows a section of the applicator 1, in which handle and fastening means are emphasized. The rod 2 is inserted fully into the applicator 1 such that just the handle 21 remains visible, The pins 23, which are fastened to the guide sleeve, are fixed in place in the L-shaped recess 15, A second handle 11 is provided at the widening 14 to simplify the handling. By this means, the user can apply force to the rod 2 through pulling the two handles together.

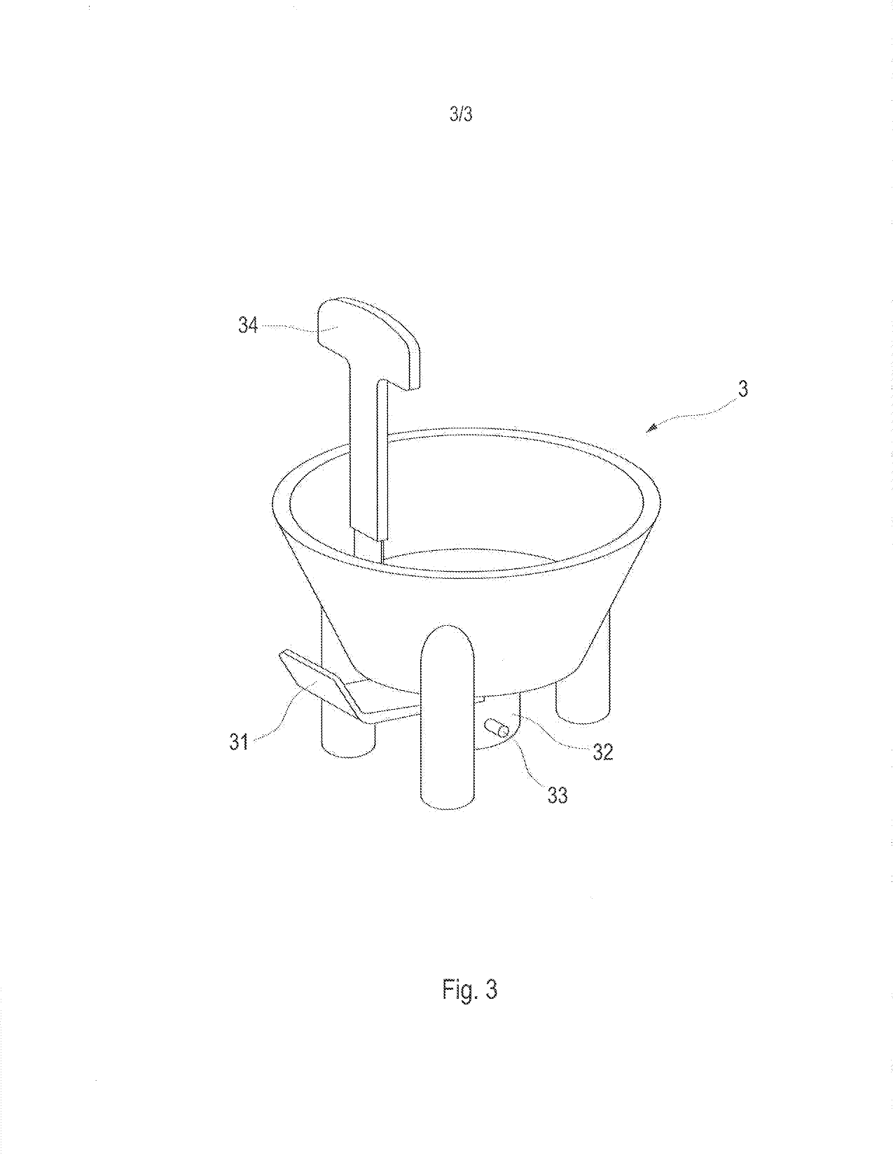

The applicator rod 12 needs to be filled first before assembling it with the rod 2. For this purpose, it is being connected to a mixing container or mixing cup 3 like the one shown in FIG. 3. The mixing cup 3 comprises fastening means 33 that is identical to the rod 2, in the present case this is a pin for a bayonet closure, by means of which the mixing cup 3 can be connected firmly to the applicator tube 12. The mixing cup 3 is similar to a funnel, whereby the drain opening can be closed through a slider 31. After the material to be filled in has been mixed in the mixing cup 3 through the use of a spatula 34, the slider 31 is opened and the material is squeezed through the drain opening and drain tube 32 into the tube through the use of the spatula 34.

### LIST OF REFERENCE NUMBERS

1 Applicator

11 Handle

12 Tube

13 Bladed shutter

14 Widened section

15 First fastening means, guide groove

2 Rod

21 Handle

22 Plunger

23 Second fastening means, pin

3 Mixing cup

31 Closure, slider

32 Dispensing tube

33 Third fastening means, pin

34 Mixing spatula

What is claimed is:

1. An applicator (1) for bone replacement material, comprising
a hollow tube (12) with a bladed shutter (13) at a front end and equipped with a first fastener (15) disposed on a rear end,
a rod (2) including a handle (21) on one end and a plunger (22) on the opposite end, arranged inside a guide sleeve that can be moved along the rod (2),
wherein the guide sleeve has a diameter that is smaller than or equal to the internal diameter of the tube (12), and second fastener (23) that can be connected to the first fastener (15) of the applicator (1) in reversible manner.

2. The applicator according to claim 1, wherein the hollow tube includes a widened section (14) at the rear end of the tube, and wherein the guide sleeve has a diameter smaller than or equal to the internal diameter of the widened section (14).

3. The applicator according to claim 1, wherein the first fastener (15) and the second fastener (23) together form a bayonet closure.

4. The applicator according to claim 1, wherein the first fastener (15) and the second fastener (24) together form a screw closure.

5. The applicator according to claim 1, wherein the bladed shutter (13) forms a tip.

6. The applicator according to claim 1, wherein the hollow tube (12) has an external diameter from 4 to 15 mm.

7. The applicator according to claim 1, wherein the hollow tube (12) has an external diameter from 8 to 13 mm.

8. The applicator according to claim 1, wherein the hollow tube (12) has a length, from the front end including bladed shutter (13) to the rear end, from 20 to 40 cm.

9. The applicator according to claim 1, wherein the hollow tube has a length, from the front end including bladed shutter (13) to the rear end, from 25 to 35 cm.

10. An applicator kit comprising
an applicator (1) according to claim 2, and
a mixing cup (3) having an underside, the underside includes at least one opening surrounded by a dispensing tube (32) disposed on the external side of the underside,
wherein the dispensing tube (32) has a diameter smaller than or equal to an internal diameter of the widened section (14), and comprises a third fastener (33) which is connected to the first fasten (15) of the applicator (1) in reversible manner.

11. The applicator kit according to claim 10, wherein a closure (31) is provided, suitable to close the opening of the mixing cup (3).

12. The applicator kit according to claim 11, wherein the closure (31) is a lock slider.

13. The applicator kit according to claim 10, further comprising
a mixing device (34) having on one end a two-dimensional stopper disposed perpendicular to an axis of the mixing device and a cross-sectional area which is smaller than or equal to the cross-sectional area of an opening of a dispensing tube (32) of the mixing cup.
